(19) United States
(12) Patent Application Publication
(10) Pub. No.: US 2006/0095087 A1
(43) Pub. Date: May 4, 2006

(54) Method for treating disease using motion style treatment

(76) Inventor: Jun-Sik Shin, Seoul (KR)

Correspondence Address: JONATHAN Y. KANG, ESQ.;LEE, HONG, DEGERMAN, KANG & SCHMADEKA, 14th Floor, 801 S. Figueroa Street, Los Angeles, CA, 90017-5554, US

(21) Appl. No.: 11/076,394

(22) Filed: Mar. 9, 2005

(30) Foreign Application Priority Data
Nov. 1, 2004 (KR) 2004-87898

(51) Int. Cl. A61N 1/34

(52) U.S. Cl. 607/46; 128/907

(57) ABSTRACT

The present invention relates to a method for treating diseases by stimulation of stiff muscles, ligaments, and nerves of the patient, using a motion style treatment (MST). The method comprises applying an acupuncture needle to a relevant and selected acupoint of a target site of the patient body, and motioning the muscle of the target site while maintaining the acupunctured state, so as to maximize circulation of Qi in the target site for acupuncturing and to activate the stimulation effects of the acupuncture needle on muscles, ligaments and nerves of interest, leading to maximized effectiveness of the acupuncture therapy. Therefore, the present invention is useful for treating temporomandibular joint disorder, herniated lumber disc, pain in the shoulder and arm, stiffness in the neck and/or hemiplegia.

### BACKGROUND OF THE INVENTION

1. Field of the Invention

The present invention relates to a method for treating a disease using a motion style treatment (MST), and more particularly to a method for treating a disease using a motion style treatment (MST) to cure disorders in patients suffering from temporomandibular joint disorder, herniated lumber disc, pain in the shoulder and arm, stiffness of the neck and/or hemiplegia, by stimulation of stiff muscles, ligaments, and nerves of the patient, involving applying an acupuncture needle to a relevant, selected acupoint of a target site of the patient's body, and motioning the muscle of the target site while maintaining the acupunctured state, thereby maximizing circulation of Qi (pronounced “chi” in Chinese)—the Chinese word used to describe the natural energy of the Universe, that is thought vital to the health of the mind and body of an organism, similar or analogous to subtle energy—in the target site for acupuncturing and activating the stimulation effects of the acupuncture needle on muscles, ligaments and nerves of interest, leading to maximized effectiveness of the acupuncture therapy.

2. Description of the Related Art

As is well known, most people suffer from various kinds of severe or mild diseases during their lifetime, and pay a great deal of attention to treatment and prevention of such diseases.

Recently, oriental medicine has been gaining popularity among the general public owing to scientific approach and empirical effectiveness thereof and has also acquired remarked reliability. Among various fields of oriental medicine, in order to perform acupuncture therapy, acupressure therapy or the like, it is first necessary to precisely locate meridians or meridian points (acupoints) corresponding to and affecting a disease of interest or being target sites.

For example, there are fourteen meridians and about 365 meridian points, and a chart plotted based on those meridians and meridian points is called an Anatomical Chart for Acupuncture Meridians. FIG. 1 shows the Anatomical Chart for Acupuncture Meridians.

Here, the Meridians are related to a response of Zang-fu organs (the Five Vital Organs and the Six Viscera) manifested on the epidermis of the body. The Su (Stream) point, an acupuncture point or acupoint positioned on the meridians, is called a meridian point.

The meridian point is a main site in which Qi and blood is received and stagnates on the body surface or passes therethrough in the system of the circulation pathways of meridians, and refers to a certain site for acupuncture, moxibustion or acupressure therapy in Chinese medicine. Specifically, the system of fourteen Meridians represents flow of meridian points and includes the Lung Meridian of the Hand-Taiyin, The Large Intestine Meridian of the Hand-Yangming, The Stomach Meridian of the Foot-Yangming, The Spleen Meridian of the Foot-Taiyin, The Heart Meridian of the Hand-Shaoyin, The Small Intestine Meridian of the Hand-Taiyang, The Urinary Bladder Meridian of the Foot-Taiyang, The Kidney Meridian of the Foot-Shaoyin, The Pericardium Meridian of the Hand-Jueyin, The Sanjiao (Triple Energizer) Meridian of the Hand-Shaoyang, The Gall Bladder Meridian of the Foot-Shaoyang, The Liver Meridian of the Foot-Jueyin, Conception Meridian, and Governor Meridian.

Due to development, popularity, and expansion of oriental medicine throughout society, there have been proposed many ways to treat or alleviate menstrual pain by use of acupuncture, moxibustion or acupressure therapy.

Among those therapies for treating maladies and ailments, acupuncture is based on the theory of Meridians. According to this theory, it is said that there are meridians, which are pathways of vital energy, through which Qi flows, and the respective meridians have meridian points having special functions. These meridian points are now sites for performing acupuncture therapy in Chinese medicine.

Locating the meridian points of interest and applying stimulus to the skin, using an acupuncture needle, to open occluded Qi pathways results in smooth circulation of Qi leading to assistance in blood circulation. That is, stimulus to the meridian points of interest induces regulation reactions inside the body and thereby enhances the natural healing ability, thus being capable of eliminating and preventing diseases.

However, recently, curemany practitioners are of the opinion that limited therapeutic effects are provided by use of acupuncture alone.

### SUMMARY OF THE INVENTION

Therefore, the present invention has been made in view of the above problems, and it is an object of the present invention to provide a method for curing disorders in a patient suffering from temporomandibular joint disorder, herniated lumber disc, pain in the shoulder and arm, stiffness of the neck and/or hemiplegia by stimulation of stiff muscles, ligaments, and nerves of the patient, involving applying an acupuncture needle to a relevant, selected acupoint of a target site of the patient body, and motioning the muscle of the target site while maintaining the acupunctured state, thereby maximizing circulation of Qi in the target site for conducting acupuncture and activating the stimulation effects of the acupuncture needle on muscles, ligaments and nerves of interest, leading to maximized effectiveness of the acupuncture therapy.

In accordance with an aspect of the present invention, the above and other objects can be accomplished by the provision of a method for treating a disease using a motion style treatment (MST) to cure disorders in a patient suffering from temporomandibular joint disorder, herniated lumber disc, pain in the shoulder and arm, stiffness of the neck and/or hemiplegia by stimulation of stiff muscles, ligaments, and nerves of the patient, comprising:

applying an acupuncture needle to a relevant selected acupoint of a target site on the patient; and

motioning the muscle of the target site while maintaining the acupunctured state, so as to maximize circulation of Qi in the target site for conducting acupuncture and to activate the stimulation effects of the acupuncture needle on muscles, ligaments and nerves of the target site.

Preferably, in the case of a patient suffering from severe muscle, ligament, and nerve stiffness, a motion may be applied to the needle acupunctured so as to relax muscles, ligaments and nerves, prior to a motion style treatment after acupuncturing.

Preferably, prior to performing the motion style treatment, a pretreatment process may be performed by a Tui Na (pronounced “twee-nah” in Chinese) therapy—a kind of massage and manipulation technique, which is an Oriental Body Work Therapy that has been used in far eastern countries including Korea, China and Japan, for a long time—in order to relax muscles or ligaments of affected parts.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is an Anatomical Chart for Acupuncture Meridians;

FIG. 2 shows acupoints for conducting acupuncture in order to treat a temporomandibular joint disorder of the human body in accordance with the present invention;

FIG. 3 shows acupoints for conducting acupuncture in order to treat a bulging disc disorder of the human body in accordance with the present invention;

FIG. 4 shows acupoints for conducting acupuncture in order to treat a pain in the shoulder and arm of the human body in accordance with the present invention;

FIG. 5 shows acupoints for conducting acupuncture in order to treat neck stiffness of the human body in accordance with the present invention; and

FIG. 6 shows acupoints for conducting acupuncture in order to treat hemiplegia of the human body in accordance with the present invention.

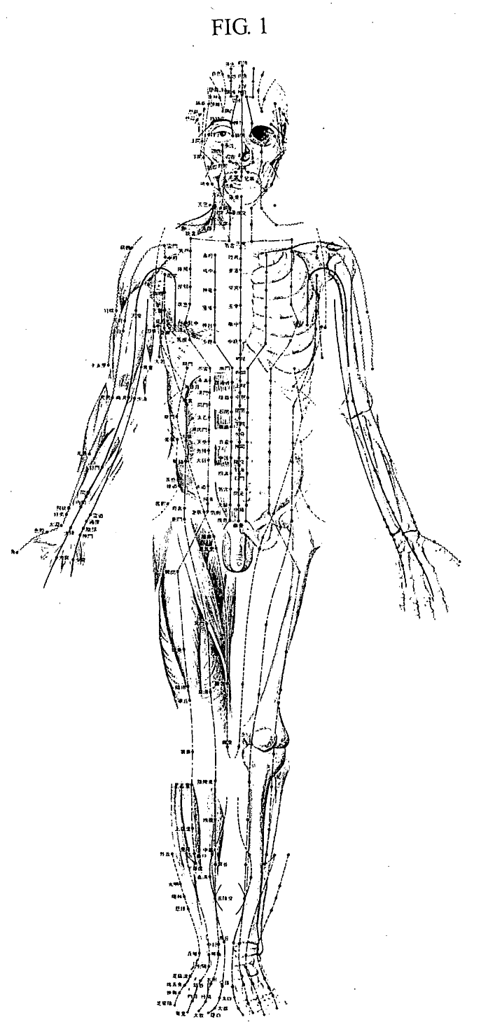

### DESCRIPTION OF THE PREFERRED EMBODIMENTS

Now, the present invention will be described in more detail.

A method for treating a disease using a motion style treatment in accordance with the present invention is a method for curing disorders in a patient suffering from temporomandibular joint disorder, herniated lumber disc, pain in the shoulder and arm, stiffness of the neck and/or hemiplegia by stimulation of stiff muscles, ligaments, and nerves of the patient, involving applying an acupuncture needle to a relevant acupoint of a target site on the patient, and motioning the muscle of the target site while maintaining the acupunctured state, thereby maximizing circulation of Qi in the target site, and activating the stimulation effects of the acupuncture needle on muscles, ligaments and nerves of interest, leading to maximized effectiveness of the acupuncture therapy.

The treatment method using such motion style treatment may further enhance therapeutic effects, which were limited in the past by utilization of acupuncture alone, because even an expert having a great deal of experience cannot apply any suitable stimulus to muscles, ligaments and nerves by applying an acupuncture needle alone.

Typically, since a multiplicity of muscles, ligaments and nerves are distributed in bones of the human body, the motion style treatment in accordance with the present invention exhibits enhanced therapeutic effects upon diseases originating from abnormality of such muscles, ligaments and nerves as targets for acupuncture therapy.

Further, the motion style treatment in accordance with the present invention is preferably performed in combination with the Tui Na therapy that corrects distorted or sprained bones. Such Tui Na therapy is a kind of therapy that corrects distorted bones and activates motoricity of skeletal muscles, and thereby cures affected parts, having a long history of more than 2,000 years. Therefore, the Tui Na therapy has a close relationship with the motion style treatment of the present invention, and is a pretreatment therapy of the present motion style treatment stimulating the acupuncture points by the motion of the acupuncture needle and motion of the muscle itself in an acupunctured state. Therefore, the Tui Na therapy is a process to treat bones and skeletal muscles.

Needless to say, after acupuncturing without use of the Tui Na therapy, it is possible to directly perform the motion style treatment, which is a method that imparts motion properties to the site of the acupuncture points of interest, but if the pretreatment process is omitted patients may experience pain during the motion style treatment.

Therefore, it is preferred to perform the Tui Na therapy in combination with the motion style treatment, from the standpoint of alleviating pain of the patient and relaxing muscles prior to treatment.

### EXAMPLES

Now, the present invention will be described in more detail with reference to the following Examples. These examples are provided only for illustrating the present invention and should not be construed as limiting the scope and sprit of the present invention.

### Example 1

Significantly different from conventional acupuncture therapy, a method for treating a disease using a motion style treatment in accordance with the present invention can realize maximum effects of the acupuncture therapy by applying an acupuncture needle to an acupoint corresponding to a disease of interest and then, shaking the needle or giving motion property to the muscle corresponding to the disease of interest immediately after acupuncturing without leaving the acupunctured needle in place for several minutes, so as to enable the acupunctured needle to stimulate the muscle, ligament and nerves of the acupoint site, thus maximizing the effectiveness acupuncture therapy.

In this example, a method for treating a disease using a motion style treatment in accordance with the present invention is applied to a patient suffering from temporomandibular joint disorder.

Generally, in patients suffering from temporomandibular joint disorder, stiffening of muscles, nerves and ligaments responsible for the motion of the temporomandibular joint causes development of disorders with symptoms observable by the fact that patients suffering from temporomandibular disorder are unable to open their mouth to the same degree as normal persons. This is because they experience smaller activity of the joints to open the mouth than normal persons, due to impaired temporomandibular joints.

Therefore, in the case of patients suffering from temporomandibular joint disorders, the Tui Na therapy is first conducted as a pretreatment process prior to the motion style treatment.

In this connection, the Tui Na therapy performs correction of cervical vertebrae by stimulating the cervical vertebrae connected with temporomandibular joints and thereby relaxing muscles of cervical vertebrae prior to conducting acupuncture therapy.

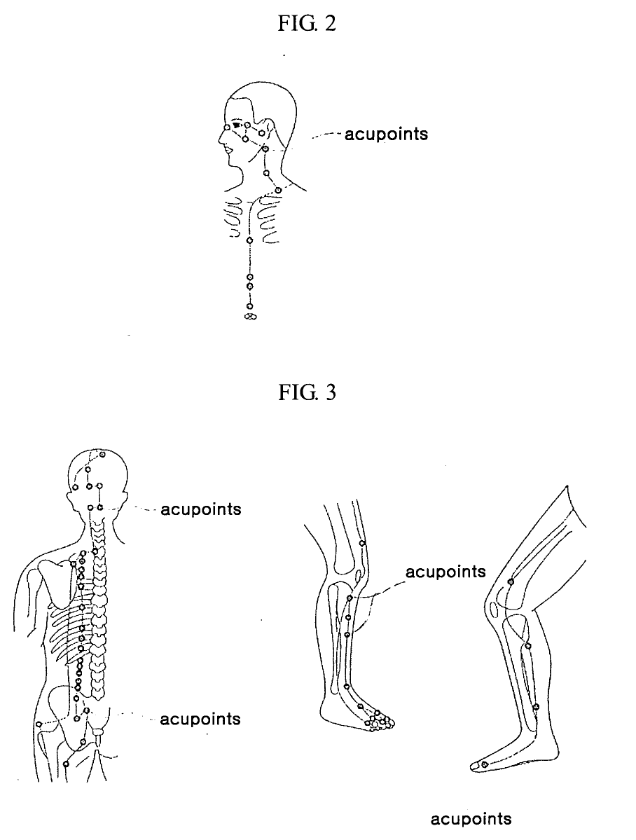

This is followed by acupuncturing at the acupoints of the temporomandibular joints while the patient suffering from temporomandibular joint disorder keeps their mouth open, as shown in FIG. 2.

The patient's mouth is motioned upward and downward while maintaining the acupunctured state. That is, the patient is instructed to repeatedly and continuously open and close their mouth.

In accordance with this motion style treatment, the acupuncture needle applied to the acupoints of the temporomandibular joints imparts direct stimulus not only to the acupoints and but also to muscles, nerves and ligaments in the vicinity of the acupoints of interest. Repeated and continuous motion corrects the temporomandibular joints. Typically, in the case of patients suffering from temporomandibular joint disorder, a single instance of such therapy is not sufficient for complete curing and thus correction of the temporomandibular joint disorder is completely effected by performing the motion style treatment for about 1 hour a day over a period of about 3 days.

### Example 2

In this example, a method for treating disease using a motion style treatment in accordance with the present invention is applied to a patient suffering from a bulging disc.

Generally, in patients suffering from a bulging disc, stiffening of muscles, nerves and ligaments governing the motion of the waist, nerve herniation between discs, or distorted shaft of discs causes development of disorders.

Also, in the case of the patient suffering from the bulging disc, a pretreatment process of the motion style treatment is preferably performed through the Tui Na therapy. At this time, for the patient suffering from the bulging disc bulging disc, the Tui Na therapy should be applied in a variety of different ways depending on conditions of the patient and location of disc bulging.

For conducting the Tui Na therapy, the patient suffering from the bulging disc lays down. Then, the Tui Na therapy is performed to correct cervical vertebrae by first turning the neck of the patient to the left and right while pressing the cervical vertebrae. After correcting the position of the cervical vertebrae by repeating this motion several times, shoulders of the patient are fixed, and the knees are held together and then raised.

Subsequently, the knees of the patient are motioned up and down. Thereby, stimulus is applied to the Musculus Obliquus Externus Abdominis, Musculus Erector Spinae and Musculus Obliquus Internus Abdominis, thus correcting the position of the lumbar vertebrae. More active correction of the position of the lumbar vertebrae may be performed by twisting the lumbar vertebrae to the right and left in the state of slight raise of the waist.

Thereafter, acupuncture is conducted upon acupoints of lumbar vertebrae, acupoints of the instep of the foot, calf of the leg and the femoral region, and acupoints of the cervical vertebrae, as shown in FIG. 3.

After the acupuncture needles are inserted, the patient is instructed to stand up and walk about while swinging both arms. The patient initially complains of pain in the waist, upon walking, but after a brief period of time, ligaments, muscles, and nerves, in the vicinity of the acupoints of interest, are stimulated by the acupuncture needle, thus resulting in curing of bulging disc.

Similarly, patients suffering from stenosis can also be cured using the method of Example 2.

Generally, stenosis is a condition in which nerves distributed within a certain disc formed in the lumbar vertebrae, unlike those of a normal person, are herniated through the corresponding disc and in this state, the nerves are constricted. In this case, the patient complains of pain due to friction between nerves of the waist and disc.

### Example 3

In this example, a method for treating a disease using a motion style treatment in accordance with the present invention is applied to a patient suffering from pain in the shoulder and arm.

Generally, in patients suffering from pain in the shoulder and arm, stiffening of muscles, nerves and ligaments of the shoulder responsible for the motion of arms leads to the development of disorders.

Also, in the case of patients suffering from pain in the shoulder and arm, a pretreatment process of the motion style treatment is preferably performed through the Tui Na therapy. Performing the Tui Na therapy for the patient suffering from pain in the shoulder and arm, the patient experiences difficulty in raising either of two arms higher. The patient will complain of pain in the shoulder when a doctor or other person attempts to forcibly raise the affected limb.

The Tui Na therapy applied to patients suffering from pain in the shoulder and arm consists of correction of cervical vertebrae and motion of the arms. This is followed by application of the acupuncture needle to acupoints of interest and then the motion style treatment, resulting in curing of disorders. Therefore, cervical vertebrae are first stimulated to correct muscles connected with shoulders and distorted cervical vertebrae.

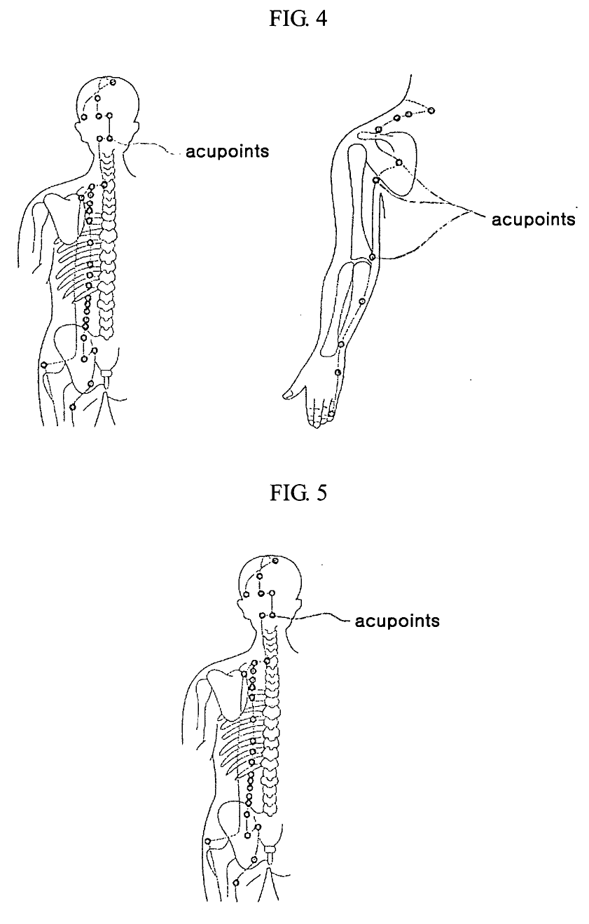

In this state, the affected limb is moved up and down. After repetition of this motion several times, acupoints of cervical vertebrae, and shoulders and arms were acupunctured, as shown in FIG. 4.

Then, the patient is instructed to continuously motion the affected limb up and down. At this time, it is preferable to pull on the patient's arms in combination with the above-mentioned motion.

Thereby, ligaments, muscles and nerves in the vicinity of acupoints of interest are stimulated by the acupuncture needle, resulting in curing of the pain in the shoulder and arm. In fact, the patient suffering from pain in the shoulder and arm could raise his arms higher after several minutes.

### Example 4

In this example, a method for treating a disease using a motion style treatment in accordance with the present invention is applied to a patient suffering from neck stiffness.

Generally, in patients suffering from neck stiffness, stiffening of muscles, nerves and ligaments of shoulders governing the motion of the back part of the neck, i.e., the upper section of cervical vertebrae, causes development of disorders.

Again, in the case of the patient suffering from neck stiffness, a pretreatment process of the motion style treatment is preferably performed through the Tui Na therapy. The Tui Na therapy is applied to the patient suffering from neck stiffness by instructing the patient to move their neck to the left and right, or assisting the patient in doing the same. Patients suffering from neck stiffness have a great deal of difficulty in motioning their neck to the right and left and it is generally difficult to motion the neck to the right and left to any substantial degree. If the patient's neck is forcibly turned, the patient will complain of neck pain.

The Tui Na therapy in the patient suffering from neck stiffness includes correction of cervical vertebrae and motion of the neck. This is followed by application of the acupuncture needle to acupoints of interest and then the motion style treatment, so as to cure disorders of the patient. Therefore, cervical vertebrae are first stimulated to correct muscles connected with the neck and distorted cervical vertebrae.

In this state, acupoints situated in the cervical vertebrae and a connection part between cervical vertebrae and the neck are acupunctured, as shown in FIG. 5.

Then, the patient is instructed to continuously motion their neck to the left and right, or the patient is assisted to perform the same. At this time, it is preferable that the patient's neck be pulled upwards or hung forward while the patient performs the above-mentioned motion.

Thereby, ligaments, muscles, and nerves in the vicinity of acupoints of interest are stimulated by the acupuncture needle, and thus the neck stiffness is cured. In fact, the patient suffering from neck stiffness will experience complete recovery after several days.

### Example 5

In this example, a method for treating a disease using a motion style treatment in accordance with the present invention is applied to a patient suffering from hemiplegia.

Generally, hemiplegia is seen in patients who suffered a cerebral hemorrhaging, or palsy several years prior to the onset of hemiplegia. That is, hemiplegia is a condition in which muscles or nerves in a portion of cervical vertebrae are paralyzed due to cerebral hemorrhaging or palsy, and then, in general, long-term disuse of the corresponding nerves or muscles for several years causes deepened muscle stiffness of the relevant parts.

Again, in the case of the patient suffering from hemiplegia, a pretreatment process of the motion style treatment is preferably performed through the Tui Na therapy. In the Tui Na therapy applied to a patient suffering from hemiplegia, the patient's fingers or wrists should be stretched by repeatedly contracting and rotating the finger/wrist. In this connection, acupuncture and motion style treatment are conducted in combination.

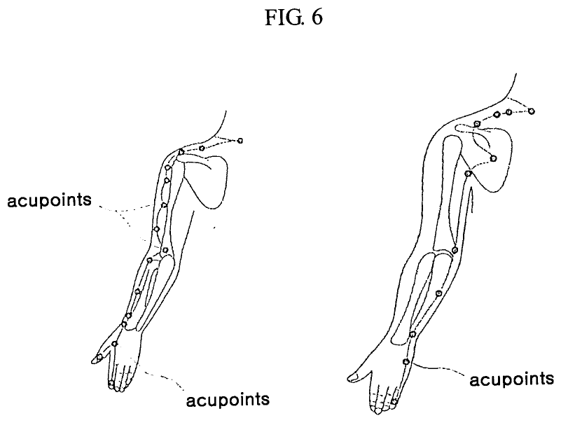

Acupuncture is generally performed on Hegu, a source point of the large intestine meridian and Shuidao, a stomach meridian of Foot-Yangming, as shown in FIG. 6. This is followed by repeated motion of sequentially stretching out from the finger to the wrist several times. Then, the vicinity of acupoints of interest is stimulated by the acupuncture needle, and thereby fingers and wrists are stretched out.

Further, the motion of stretching out the affected limb is continuously repeated following acupuncturing acupoints situated on the arm. In particular, patients suffering from hemiplegia exhibit severe muscle stiffness of half the body due to long-term disuse thereof, for a period of several years, and thus experience difficulty such as complaining of pain in acupuncture operation. However, the motion style treatment should be repeated with patience after acupuncturing acupoints of interest.

In addition, with regard to a method for treating disease using a motion style treatment in accordance with the present invention, when the patient suffers from severe stiffness of muscles, ligaments and nerves, preferably, motion is first given to the acupunctured needle so as to relax muscles, ligaments and nerves of acupoint sites after acupuncturing prior to performance of the motion style treatment, and then the patient is instructed to carry out the motion style treatment on their own.

As described above, conventional acupuncture therapy provides a method of treating a disease by conducting acupuncture and then leaving the acupunctured needle in place for a long period of time, but does not effectively stimulate acupoints of interest, and also muscles, nerves and ligaments in the vicinity of the acupoints. However, the motion style treatment in accordance with the present invention has the advantage of providing a rapid cure by continuously motioning muscles around or in the vicinity of the acupuncture needle, after acupuncturing, in combination with the Tui Na therapy, thus equally stimulating muscles, nerves and ligaments to activate functions thereof.

Although the preferred embodiments of a method for treating disease using a motion style treatment in accordance with the present invention have been disclosed for illustrative purposes, those skilled in the art will appreciate that various modifications, additions and substitutions are possible, without departing from the scope and spirit of the invention as disclosed in the accompanying claims.

What is claimed is:

1. A method for treating a disease using a motion style treatment to cure disorders in a patient suffering from temporomandibular joint disorder, herniated lumber disc, pain in the shoulder and arm, neck stiffness and/or hemiplegia, by stimulation of stiff muscles, ligaments, and nerves of the patient, comprising:
applying an acupuncture needle to a relevant, selected acupoint of a target site of the patient body; and
motioning the muscle of the target site while maintaining the acupunctured state, so as to maximize circulation of Qi in the target site for acupuncturing and to activate the stimulation effects of the acupuncture needle on muscles, ligaments and nerves of the target site.

2. The method as set forth in claim 1, wherein in the case of a patient suffering from severe muscle, ligament, and nerve stiffness, the motion is applied to the acupunctured needle so as to relax muscles, ligaments and nerves of acupoint sites, prior to a motion style treatment after acupuncturing.

3. The method as set forth in claim 1, wherein prior to performing the motion style treatment, a pretreatment process is performed by a Tui Na therapy so as to relax muscles or ligaments of affected parts.
